(19) United States
(12) Patent Application Publication
(10) Pub. No.: US 2018/0311709 A1
(43) Pub. Date: Nov. 1, 2018

(54) LUMEN CLEARING DEVICES

(71) Applicant: TELEFLEX MEDICAL INCORPORATED, Morrisville, NC (US)

(72) Inventors: David Troy ROWE, Sinking Spring, PA (US); Rodney Wilmer DENLINGER, Lancaster, PA (US); John C. VICTOR, Kunkletown, PA (US)

(21) Appl. No.: 15/771,049

(22) PCT Filed: Oct. 26, 2016
(86) PCT No.: PCT/US16/58750

§ 371 Date: Apr. 25, 2018

Related U.S. Application Data
(60) Provisional application No. 62/246,403, filed on Oct. 26, 2015.

(51) Int. Cl. B08B 9/045; A61B 90/70; A61M 16/04

(52) U.S. Cl. CPC B08B 9/045; A61B 90/70; A61M 16/047; A61M 2209/10; A61M 16/0463; A61B 2090/701; B08B 2209/04; A61M 16/0447

(57) ABSTRACT

The present disclosure relates to a lumen clearing device for a lumen of an airway device and, in particular, an endotracheal tube. The lumen clearing device may include a shaft and a ridge disposed along a distal portion of the shaft, where the ridge extends from the shaft in a spiral configuration. The lumen clearing device may further include a rotating mechanism connected to a proximal portion of the shaft that is configured to rotate the shaft and the ridge. The shaft may be configured to be inserted into a lumen of an airway tube. The ridge, when rotated as the shaft is inserted into the lumen of the airway tube, may contact and convey a material disposed in the lumen of the airway tube out of the lumen of the airway tube.

### TECHNICAL FIELD

This application relates generally to a device for clearing an interior of medical device and, more specifically, to auger assemblies for clearing a lumen of an airway tube and related advancement devices for auger assemblies.

### BACKGROUND ART

Airway devices, such as, for example, endotracheal tubes, have been used to facilitate artificial ventilation of anesthetized patients and non-surgical unconscious patients with compromised spontaneous respiration. Endotracheal tubes typically comprise a flexible airway tube that is curved between its distal and proximal ends. Endotracheal tubes may be inserted into a patient through an upper airway passageway (e.g., oral cavity). Once positioned in a patient, the distal end of the endotracheal tube may be proximal to a carina of the trachea, and a proximal end of the endotracheal tube may extend a certain distance outside of the mouth of the patient. Endotracheal tubes may be used in a variety of applications ranging from durations of less than an hour, such as, for example, for surgical patients, to durations lasting up to and occasionally exceeding seven (7) days, such as, for example, for compromised unconscious non-surgical patients.

During longer duration or chronic intubations, the central space of a lumen of an endotracheal tube may become occluded by various biomaterials, including, for example, lung and bronchial secretions of hydrogels such as sputum and mucus. These biomaterials may include polysaccharide matrices and be highly adhesive in nature. Accordingly, they may deposit and adhere to an interior surface of an airway tube of an endotracheal tube. The biomaterial deposits may form the foundation for biofilm formations of pathogenic strains of bacteria. Such may pose a health risk to the patient as well as diminish the effectiveness of the ventilating function of the device. In as little as eight (8) hours post-intubation, the lumen of an endotracheal tube may become significantly occluded, resulting in increased work of breathing.

Various methods have been developed to remove and limit the build-up of occlusive material in an airway device such as an endotracheal tube. One traditional approach to endotracheal tube lumen maintenance involves the use of an intraluminal suction catheter by which occlusive material within the lumen is aspirated from the interior of the lumen. Another more recent approach involves the use of a balloon mechanism that is attached to a distal end of an intraluminal catheter. The intraluminal catheter may be inserted into the lumen of the endotracheal tube, and the balloon mechanism may be inflated such that it presses against an inner wall of the lumen. Once the balloon mechanism is inflated, the catheter including the balloon mechanism may be extracted, whereby the balloon, as it traverses the endotracheal tube lumen from its distal to its proximal end, may scrape against the inner wall of the lumen to displace the occlusive material that is disposed on the wall. The displaced occlusive material may then exit the lumen of the endotracheal tube in advance of the balloon mechanism at the proximal end of the endotracheal tube.

While such approaches may provide for the removal of occlusive material within an airway device such as an endotracheal tube, they are associated with a number of negative aspects. For example, both approaches involve the insertion of a catheter or other mechanism a distance beyond certain occlusive material that may be disposed in the endotracheal tube lumen. In particular, both approaches require that an end of a catheter is inserted beyond a first portion of the endotracheal tube lumen in which occlusive material may be disposed. Accordingly, these methods may risk the dislodgement of occlusive material during their insertion process. Such may represent an aspiration hazard. If occlusive material is dislodged from a distal tip of the endotracheal tube, then such material may be aspirated into the bronchia or lungs of the patient. The occlusive material in the lumen of the endotracheal tube may also contain significant pathogenic loading and may therefore lead to a respiratory infection such as, for example, ventilator acquired pneumonia.

Accordingly, there is a need for a lumen clearing device that mitigates the risk of dislodging occlusive material in a lumen of an airway device as the lumen clearing device traverses the lumen from its proximal to distal ends.

### SUMMARY OF THE DISCLOSURE

The foregoing needs are met, to a great extent, by a lumen clearing device that includes a mechanism configured to capture occlusive material as the lumen clearing device traverses a length of a lumen.

In an aspect, a lumen clearing device for an airway tube comprises an auger assembly. The auger assembly includes a shaft and a ridge disposed along a distal portion of the shaft, wherein the ridge extends from the shaft in a spiral configuration. The lumen clearing device further includes a rotating mechanism connected to a proximal portion of the shaft. The shaft is configured to be inserted into a lumen of the airway tube, and the rotating mechanism is configured to rotate the shaft and the ridge. The ridge of the device, when rotated, is configured to contact and convey a material disposed in the lumen of the airway tube out of the lumen of the airway tube.

In another aspect, a lumen clearing device for an airway tube comprises a manual advancement device, which is designed to rotate and advance an auger assembly. The manual advancement device may include one or more of: a handle, an advancer, thrust bearing components, an advancer tube, locking components, adaptors, and a sheath. The advancer may be attached to the auger assembly and may slide over the advancer tube in order to advance the auger assembly into the lumen of the airway tube such as an endotracheal device. The thrust bearing components may attach the auger assembly to the advancer but allow the auger assembly to rotate as it is advanced into the lumen of the airway tube. The sheath may be held between two different locking components, which may be released from their locking position to allow the sheath to expand when the auger assembly is being removed from the lumen of the airway tube.

In yet another aspect, a lumen clearing device for an airway tube comprises an auger assembly and a manual advancement device. The auger assembly may comprise a shaft and a ridge disposed along a portion of the shaft, wherein the ridge extends from the shaft in a helical configuration. The manual advancement device may comprise a tubular portion; an advancer portion disposed on the tubular portion and configured to axially displace along the tubular portion; and a first adaptor portion configured to rotate the auger assembly. The auger assembly initially may be disposed within the tubular portion. The advancer portion may axially displace the auger assembly to insert the auger assembly into a lumen of the airway tube when the advancer portion axially displaces along the tubular portion. The auger assembly may rotate when the advancer portion axially displaces the auger assembly to contact and convey a material disposed in the lumen of the airway tube out of the lumen of the airway tube.

There are, of course, additional aspects of the disclosure that will be described below and which will form the subject matter of the claims. In this respect, before explaining at least one aspect of the disclosure in detail, it is to be understood that the disclosure is not limited in its application to the details of construction and to the arrangements of the components set forth in the following description or illustrated in the drawings. The disclosure is capable of aspects in addition to those described and of being practiced and carried out in various ways. Also, it is to be understood that the phraseology and terminology employed herein, as well as the Abstract, are for the purpose of description and should not be regarded as limiting.

As such, those skilled in the art will appreciate that the conception upon which this disclosure is based may readily be utilized as a basis for the designing of other structures, methods, and systems for carrying out the several purposes of the disclosure. It is important, therefore, that the claims be regarded as including such equivalent constructions insofar as they do not depart from the spirit and scope of the disclosure.

### BRIEF DESCRIPTION OF THE DRAWINGS

In order that the disclosure may be readily understood, aspects of the disclosure are illustrated by way of examples in the accompanying drawings.

FIG. 1 is a perspective view of a first exemplary lumen clearing device for an airway device according to an aspect of the disclosure.

FIG. 2 is an enlarged view of a distal portion of the lumen clearing device depicted in FIG. 1.

FIG. 3 depicts components of a second exemplary lumen clearing device for an airway device prior to assembly showing the attachment of each component to the others according to an aspect of the disclosure.

FIG. 4 depicts a sectional view of an auger of the lumen clearing device depicted in FIG. 2.

FIG. 5 depicts the lumen clearing device depicted in FIG. 2 extracted from an airway device according to an aspect of the disclosure.

FIG. 6 depicts a third exemplary lumen clearing device for an airway device including an auger assembly and a manual advancement device for advancing the auger assembly according to an aspect of the disclosure.

FIG. 7 depicts the third exemplary lumen clearing device depicted in FIG. 6 with the auger assembly advanced into the airway device according to an aspect of the disclosure.

FIG. 8 depicts an enlarged view of the manual advancement device of the third exemplary lumen clearing device depicted in FIG. 6.

FIG. 9 depicts a deconstructed view of a distal end of the manual advancement device of the third exemplary lumen clearing device depicted in FIG. 6.

FIG. 10 depicts an enlarged view of the distal end of the manual advancement device of the third exemplary lumen clearing device depicted in FIG. 6.

FIGS. 11 and 12 depict different enlarged views of an adapter component of the manual advancement device of the third exemplary lumen clearing device depicted in FIG. 6.

FIG. 13 depicts an enlarged view of a proximal end of the manual advancement device of the third exemplary lumen clearing device depicted in FIG. 6 with an advancer component of the manual advancement device depicted transparently to show inner components of the manual advancement device.

FIGS. 14 and 15 depict different enlarged views of the proximal end of the manual advancement device of the third exemplary lumen clearing device depicted in FIG. 6 with the advancer component of the manual advancement device removed.

FIG. 16 depicts an enlarged view of the proximal end of the manual advancement device of the third exemplary lumen clearing device depicted in FIG. 6 with a handle component of the manual advancement device removed.

FIG. 17 depicts a fourth exemplary lumen clearing device for an airway device including an auger assembly and a manual advancement device for advancing the auger assembly according to an aspect of the disclosure.

FIG. 18 depicts the fourth exemplary lumen clearing device depicted in FIG. 17 with the auger assembly advanced beyond a distal end of the lumen clearing device according to an aspect of the disclosure.

FIG. 19 depicts a deconstructed view of a distal end of the manual advancement device of the fourth exemplary lumen clearing device depicted in FIG. 17.

Aspects of a lumen clearing device according to aspects of the disclosure are described with reference to the drawings, in which like reference numerals refer to like parts throughout.

### DETAILED DESCRIPTION

Systems and methods disclosed herein provide a lumen clearing device that reduces the risk of dislodging occlusive material disposed within a lumen of an airway device while the lumen clearing device traverses through the lumen. As such, systems and methods disclosed herein minimize aspiration hazards as well as the potential for respiratory infection.

Systems and methods disclosed herein may implement a compliant auger mechanism or assembly. The auger mechanism may be rotated as it is inserted into a lumen of an airway device, such as, for example, an endotracheal tube, a tracheostomy tube, or a laryngeal mask airway tube. The auger mechanism may be rotated at a rate equal to or exceeding a rate of linear advancement of the auger mechanism. In an aspect, once the auger mechanism is fully inserted into the lumen of the airway device (e.g., once the auger mechanism has traversed the full length of the lumen of the airway device such that its distal end is coplanar with a distal end of the airway device), the rotation of the auger mechanism may be halted. The auger mechanism may then be extracted from the lumen of the airway device, whereby an outer edge of the auger mechanism may scrape against and remove occlusive material from an inner wall of the lumen of the airway device. In an alternative aspect, the auger mechanism may continue to be rotated as it is extracted from the lumen of the airway device. In this alternative aspect, the outer edge of the auger mechanism may also be configured to contact and remove occlusive material from the interior of the lumen of the airway device.

The auger mechanism may be constructed from a single polymer flute (e.g., a silicone flute) that is helically formed (e.g., formed in a spiral fashion) about a torsionally rigid shaft. The body of the flute may be formed of a soft durometer material that is capable of deforming in compliance with an internal dimension and cross-sectional shape of the airway device. For example, in the case of an endotracheal tube, the body of the flute may be sufficiently soft to conform to an interior space of the flexible tube of the endotracheal tube. The edge of the flute distal to the shaft may be constructed of a more rigid and less compliant material such that it is capable of shearing against an interior wall of the lumen of the airway device both when the auger mechanism is rotating or during a non-rotational extraction of the auger mechanism from the airway device.

According to certain aspects of the disclosure, the shaft and flute assembly (e.g., the auger mechanism) may be rotated by a mechanical coupling to an electric motor and drivetrain assembly or by any other suitable mechanical or electrical means. For example, as an alternative to using an electric motor, the shaft and flute assembly may be rotated by hand, such as, for example, by using a hand-operated lever or handle. In certain aspects, the auger mechanism may also include a sheath configured to receive, contain, and collect material that is ejected from a distal end of the airway device as the auger mechanism is rotated or extracted from the lumen of the airway device. The auger mechanism described herein may be designed for one-time use but may also utilize certain components (e.g., an electric drive element) that are detachable from the single-use portions and are reusable in multiple applications. Such reusable components would be compatible with re-sterilization methods or be isolated from the patient by means of a sterile container (e.g., an envelope or bag).

According to other aspects of the disclosure, the shaft and flute assembly (e.g., the auger mechanism) may be advanced and rotated using a manual advancement device. The manual advancement device may provide an effective low cost method for advancing the auger into the airway device and capturing removed material (e.g., mucus and sputum) upon extraction. In an embodiment, a tube adaptor may be attached to an airway device such as an endotracheal tube. The tube adaptor includes a distal end that is connected to a proximal end of the endotracheal tube. The tube adaptor may include a port that is at an angle of ninety (90) degrees relative to a longitudinal axis of the tube adaptor. The 90-degree port allows for the attachment of a ventilator line for uninterrupted therapy. The tube adaptor also includes a proximal end with an opening. When the manual advancement device is being used for secretion removal, the manual advancement device may be attached to the proximal end of the tube adaptor. When the manual advancement device is not attached to the tube adaptor, the opening at the proximal end of the tube adaptor may be closed with an end cap.

The manual advancement device may include a handle for an operator to grasp and hold the device in free space. The handle may be locked onto an advancer tube. The advancer tube may be a rigid or flexible tube that guides the advancer. The manual advancement device may also include an advancer that is disposed on the advancer tube and is designed to slide over the advancer tube. The advancer tube may house an auger that is designed to be inserted or passed into the endotracheal tube in order to carve through material (e.g., mucus and sputum) in a helical pattern. The auger may be constructed of a polymer or silicone flute that is helically formed about a torsionally rigid shaft. The advancer may include a portion that resides within the advancer tube that grasps or holds onto thrust bearing components. The thrust bearing components may include a thrust bearing disk that is attached to a proximal end of the auger and two thrust bearings that are disposed on each side of the thrust bearing disk. The thrust bearings may hold the thrust bearing disk in place but be made from a low coefficient of friction material, thereby allowing the thrust bearing disk to freely rotate relative to the thrust bearings. A thrust bearing disk retains the thrust bearings and the thrust bearing disk within the advancer such that the advancer is effectively attached to the auger.

The advancer may be slid over the advancer tube to insert the auger into the endotracheal tube. Due to the arrangement of the thrust bearing components and their attachment to the advancer, the auger may slide forward with the advancer but is also free to spin or rotate relative to the advancer. The advancer may insert the auger into the endotracheal tube such that the auger traverses the full length of the endotracheal tube. More specifically, the advancer may insert the auger into the endotracheal tube until a distal end of the auger is disposed at a distal end of the endotracheal tube.

The manual advancement device may further include an adaptor cap that is secured to a distal end of the advancer tube and acts as a positive stop for the advancer. When the advancer is fully displaced along the length of the advancer tube, the adaptor cap may snap into a slot formed on the advancer, thereby locking the advancer to the adaptor cap together. In particular, the adaptor cap may have a tab that engages with a slot formed in the advancer when the advancer is fully deployed. The adaptor cap may also have an O-ring groove for retaining one end of a sheath. Specifically, the adaptor cap may have a groove for receiving an O-ring (e.g., a loop of elastomer), which can be used to fasten one end of a sheath to the adaptor cap. The adaptor cap further has an internal helical groove that is shaped to receive the auger as it is pushed through the distal end of the manual advancement device and inserted into the endotracheal tube. The helical groove may be designed to rotate the auger as it is pushed through the groove and into the endotracheal tube. The manual advancement device may also include an adaptor cap lock that is attached to (e.g., snapped onto) the adaptor cap. The adaptor cap lock may lock movement of the adaptor cap relative to an adaptor until the advancer is locked onto the adaptor cap. In particular, the adaptor cap lock may include a retaining arm that holds onto a portion of the adaptor such as a boss disposed on the adaptor. When the advancer is locked onto the adaptor cap, the advancer may displace the retaining arm, thereby releasing the adaptor and allowing the adaptor cap to move relative to the adaptor.

According to an aspect, the adaptor may be keyed to a channel or slot formed in the adaptor cap. When the retaining arm is displaced and the adaptor is released, the adaptor cap may need to be turned or rotated relative to the adaptor in order to separate the adaptor cap from the adaptor. In particular aspects, the adaptor cap and certain other components disposed proximate to the adaptor cap (e.g., the advancer tube, the auger, the handle) may be turned 180 degrees and removed from the adaptor. The 180-degree rotation allows the distal end of the auger to sweep its final advanced location in effort to capture any remaining mucus and sputum at a distal end of the endotracheal device. Once rotated, the assembly including the auger, the adaptor cap, the adaptor cap lock, the advancer tube, the advancer, the handle, and thrust bearing members may be pulled from the adaptor and endotracheal tube.

The manual advancement device may include a sheath formed of a loose fitting transparent material. The sheath includes a first end that may be fastened to the adaptor cap and a second end that may be fastened to the adaptor. The sheath may be fastened using O-rings, which fit into grooves on the adaptor cap and the adaptor. The sheath may be bunched together when the adaptor cap and the adaptor are connected. But when the adaptor cap and other components are removed from the endotracheal tube, leaving the adaptor attached to the tube adaptor, the separation of the adaptor cap from the adaptor expands and straightens the sheath such that it captures the auger and the material (e.g., mucus, sputum) that the auger removes from the endotracheal tube. Once the auger is fully retracted from the endotracheal tube, the adaptor may be disengaged from the tube adaptor, and the end cap may be placed once again on the proximal end of the tube adaptor to allow the endotracheal tube to be used normally. The disengaged manual advancement device and auger may be discarded.

According to another embodiment, the manual advancement device may not include one or more locking features. Similar to the embodiment described above, a tube adaptor may reside on an endotracheal tube. The tube adaptor has a distal end that is connected to a proximal end of the endotracheal tube and a proximal end that is capped. The tube adaptor may include a 90-degree port that is attached to a ventilator. When removing secretions and other material from the endotracheal tube, the proximal end of the tube adaptor may be uncapped, and a distal end of the manual advancement device may be attached to the proximal end of the tube adaptor. The manual advancement device may include a handle for stabilizing the device in free space. While holding the handle, an advancer may be advanced to a distal position on a tubular guide. The advancer may be attached to an auger, which is helically driven into the endotracheal tube as the advancer is advanced along the tubular guide.

The manual advancement device also includes an adaptor cap and an adaptor. The manual advancement device may not have an adaptor cap lock or other locking mechanism that locks the position of the adaptor cap relative to the adaptor. The adaptor cap may include a slot that receives a protruding portion (e.g., a boss) of the adaptor. The slot may include a section that runs circumferentially around the adaptor cap and a section that runs longitudinally along the length of the adaptor cap. Once the advancer fully advances the auger into the endotracheal tube, the adaptor cap may be separated from the adaptor by rotating the adaptor cap 180 degrees (such that the protruding portion of the adaptor travels along the circumferential section of the slot to the longitudinal section of the slot) and pulling the adaptor cap away from the adaptor (such that the protruding portion of the adaptor travels down the length of the longitudinal section of the slot).

The manual advancement device may also include a sheath that is held between the adaptor cap and the adaptor. The sheath may initially be bunched between the adaptor cap and the adaptor. As the adaptor cap is separated from the adaptor during extraction of the auger from the endotracheal tube, the sheath may straighten and capture the auger and material (e.g., mucus, sputum) that the auger removes from the endotracheal tube. Once the auger is removed and captured within the sheath, the adaptor may be detached from the tube adaptor, and the tube adaptor may be recapped. The manual advancement device including the auger may be discarded.

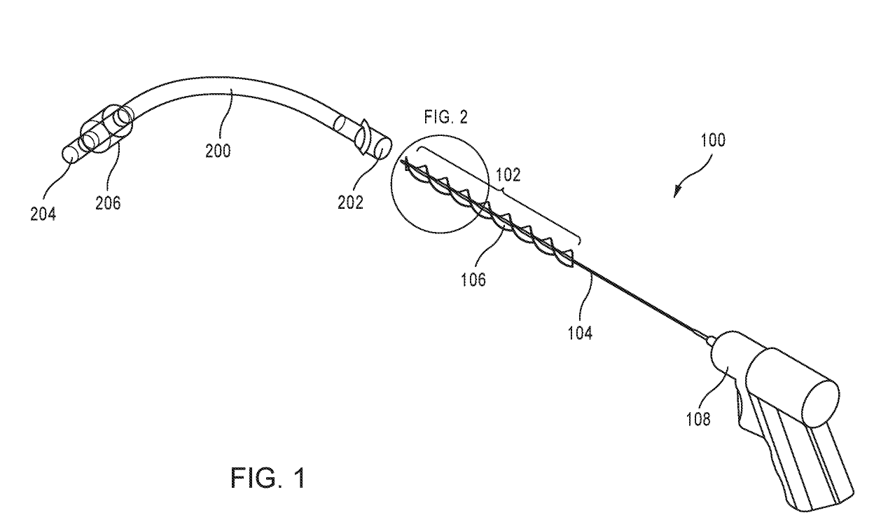

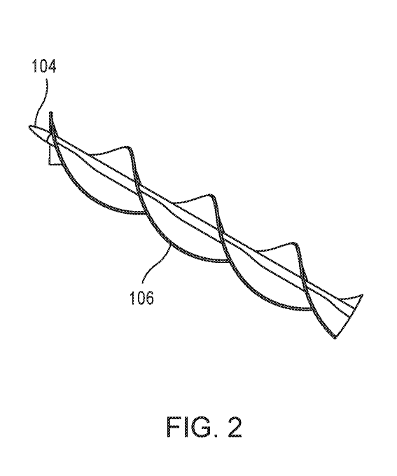

Referring to FIG. 1, an example lumen clearing device 100 and endotracheal tube 200 are depicted. The lumen clearing device 100 includes a shaft 104 and a ridge or flute 106. The flute 106 is formed in a spiral configuration on the shaft 104 along a region 102. FIG. 2 provides an enlarged view of the flute 106 formed in a spiral configuration on the shaft 104. The shaft 104 may be formed of a flexible material that is capable of deforming to a curve of the endotracheal tube 200 but is rigid in a torsional direction. As such, the shaft 104 may be formed such that it is flexible along a neutral bending axis but is torsionally rigid.

The flute 106 may be of one-piece construction but have an inner portion and an outer portion that differ in their material properties. In particular, the flute 106 may have an inner portion (e.g., a main body) that is configured to deform to an internal dimension of the endotracheal tube 200. As such, the inner portion of the flute 106 may be designed to be soft and compliant. The flute 106 may also have an outer portion that is configured to shear against an inner wall of the endotracheal tube 200. The outer portion of the flute 106 may be designed to be sufficiently rigid such that it is capable of making contact with and shearing against an inner wall of the endotracheal tube. Thus, the outer portion of the flute 106 may be designed to be more rigid than the inner portion of the flute 106.

The entire flute 106, including its inner portion and its outer portion, may be formed of a single polymer, such as, for example, silicone. In order to achieve varying rigidity throughout the flute 106 (e.g., in order to design the flute to have a compliant inner portion but a more rigid outer portion), the thickness of the flute may be varied. In particular, the inner portion of the flute 106 may be formed of less thickness than the outer portion of the flute 106.

As depicted in FIG. 1, an end of the shaft 104 that is opposite to the region 102 may be attached to a rotating mechanism 108. The rotating mechanism 108 may comprise an electric motor that is configured to rotate the shaft 104 and the flute 106 (which is mounted on the shaft 104). The rotating mechanism 108 may include a drivetrain assembly that connects the shaft 104 to the electric motor.

While the rotating mechanism 108 depicted in FIG. 1 include an electric system that is capable of rotating the shaft 104 and the flute 106, one of ordinary skill in the art would appreciate that other means of rotating the shaft 104 and the flute 106 may be utilized. For example, a mechanical handle may be attached to the end of the shaft opposite the region 102 instead of the rotating mechanism 108. The handle may be hand operated; thus, a physician or other operator may actuate the handle by exerting a force to the handle that causes the handle to move and thereby rotate the shaft 104 and the flute 106.

The endotracheal tube 200 includes a proximal end 202 and a distal end 204. The endotracheal tube 200 may be inserted into a patient (not depicted) such that the distal end 204 of the endotracheal tube 200 is disposed proximal to a carina of the patient. In particular, the distal end 204 of the endotracheal tube 200 may be disposed approximately five (5) centimeters from the carina of the patient. The endotracheal tube 200 may also include a balloon 206, which may be inflated to secure the endotracheal tube 200 in place and to prevent leakage of a fluid or gas (e.g., a stomach acid) around the endotracheal tube 200.

In operation, the lumen clearing device 100 may be inserted into the endotracheal tube 200. The rotating mechanism 108 may rotate the shaft 104 and the flute 106 of the lumen clearing device 100 as it traverses from the proximal end 202 of the endotracheal tube 200 to the distal end 204 of the endotracheal tube 200. The rotating mechanism 108 may rotate the shaft 104 and the flute 106 at a rate depending on factors including, for example, a pitch of the flute 106, a diameter of the lumen of the endotracheal tube 200, and a lateral insertion rate of the lumen clearing device 100. In an aspect, the rotating mechanism 108 may rotate the shaft 104 and the flute 106 at a rate that is equal to or greater than a rate of travel or advancement of the shaft 104 in a spiral or helical manner into the lumen of the endotracheal tube 200. Such a rate of rotation may increase the ability of the flute 106 of the lumen clearing device 100 to clear material out of the interior of the endotracheal tube 200. The flute 106 of the lumen clearing device 100 may be shaped such that it is capable of conveying occlusive material that it contacts in a direction towards the proximal end 202 of the endotracheal tube 200. As such, the region 102 of the lumen clearing device 100 having the flute 106 that is formed on the shaft 104 may function similar to an auger or screw conveyor.

The lumen clearing device 100 may be advanced a certain distance into the endotracheal tube 200 and extracted from the endotracheal tube 200. In a preferred aspect of the disclosure, the lumen clearing device 100 may be advanced until a distal end of the region 102 is coplanar with the distal end 204 of the endotracheal tube 200. Accordingly, the lumen clearing device 100 may be inserted in the endotracheal tube 200 such that it travels through the entire length of the endotracheal tube 200. The region 102 of the lumen clearing device 100, and, specifically, the flute 106 of the lumen clearing device 100, may operate to contact and convey material disposed within the endotracheal tube 200 out of the proximal end of the endotracheal tube 200 as the lumen clearing device 100 is inserted through the endotracheal tube 200. The flute 106 of the lumen clearing device 100 may also be designed to scrape against an interior wall of the endotracheal tube 200 as the lumen clearing device 100 is extracted from the endotracheal tube 200.

In certain aspects of the disclosure, the lumen clearing device 100 may additionally include a sheath (not depicted) that may be disposed proximal to the proximal end 202 of the endotracheal tube 200 such that material which is conveyed out of the proximal end 202 of the endotracheal tube 200 is collected and held in the sheath.

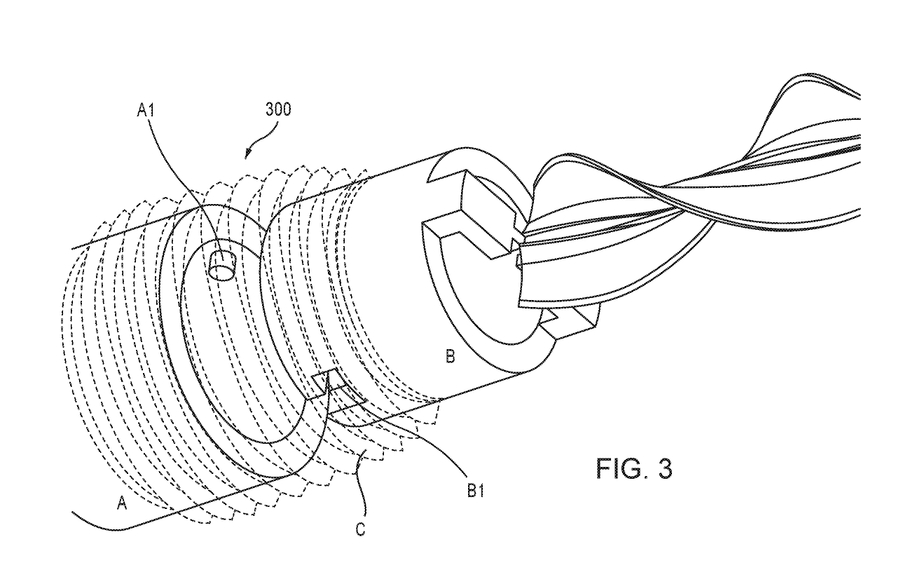

Referring now to FIGS. 3 through 5, an example lumen clearing device 300 is depicted. As depicted in FIG. 3, the lumen clearing device 300 includes a base component A, a top component B, a sheath C, and an auger D. The base component A and the top component B are permanently attached together by the sheath C.

The base component A includes a protrusion A1, and the top component B includes an opening B2 for receiving the protrusion A1 and locking the two components together. Specifically, the base component A and the top component B can be joined together by placing a portion of the top component B having the opening B2 within the base component A, and turning the base component A relative to the top component B such that the protrusion A1 of the base component A locks into the opening B1 of the top component B.

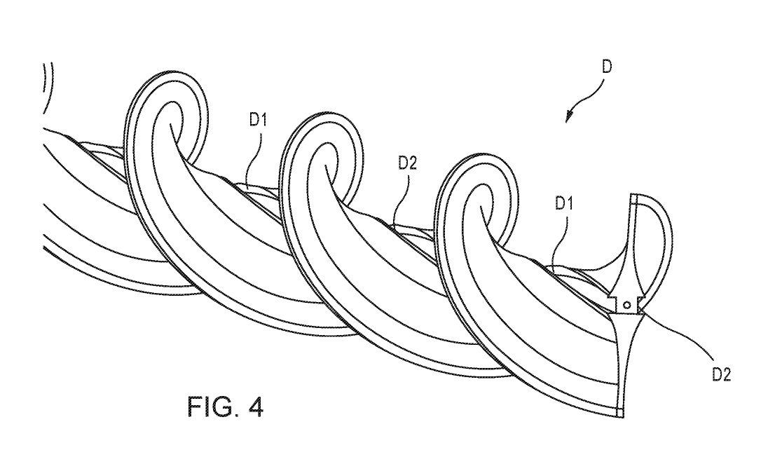

The top component B further includes two opposing guide teeth or keys. The two opposing guide teeth may be configured to fit into two corresponding key ways D1, D2 (depicted in FIG. 4) of the auger D. The key ways D1, D2 may extend along the length of the auger D and be disposed on opposite sides of a central shaft of the auger D. The key ways D1, D2 may have the same pitch as a blade or flute portion of the auger D. The auger D may be fitted into the top component B such that the two opposing guide teeth of the top component B are each positioned in a key way D1 or D2. In such a configuration, the auger D may be advanced through the top component B as the two opposing guide teeth of the top component B slide through the key ways D1, D2.

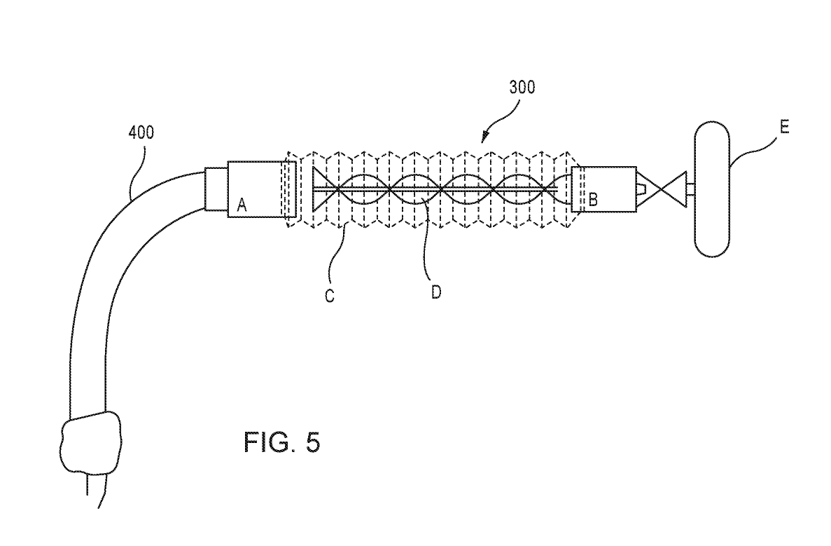

As depicted in FIG. 5, the lumen clearing device 300 may be used to clear occlusive material from an interior of an endotracheal tube or ETT 400. The base component A and the top component B of the lumen clearing device 300 may be assembled together in their lock position (i.e., the base component A and the top component B may be joined together such that the protrusion A1 of the base component A locks into the opening B1 of the top component B). The lumen clearing device 300 may be placed into a top of a fitting of the ETT 400, and the base component A may lock together with the fitting of the ETT 400 via a friction taper lock. While a taper lock is provided as an example of a connection between the lumen clearing device 300 and the ETT 400, one of ordinary skill in the art would also appreciate that other methods of locking the lumen clearing device 300 and the ETT 400 together may be used, such as, for example, a threaded mating.

The key ways D1, D2 of the auger D may be engaged with the two opposing teeth of the top component B, and the auger D may be pushed into and through the top component B such that it advanced into the ETT 400. As the auger D is advanced into the ETT 400, the auger D may rotate as the two opposing teeth of the top component B follow the paths created by the two key ways D1, D2. In certain aspects, the rotation of the auger D may also be facilitated by a spinning handle E (depicted in FIG. 5). The spinning handle E may be attached to a proximal side (i.e., a side opposite the base component A) of the auger D. The spinning handle E may be operated via a motor or by hand.

When the auger D is advanced a predetermined distance into the ETT 400 (for example, when the auger D is advanced its full length into the ETT 400), the top component B may be unlocked from the base component A. More specifically, the top component B may be turned relative to the base component A to release the protrusion A1 from the opening B1. When the top component B is unlocked from the base component A, the top component B and the auger D may be pulled away from (i.e., pulled out from) the base component A and the ETT 400. The auger D may not rotate as the top component B and the auger D are pulled away from the base component A. In particular, the rotation of the auger D may be prevented because the two opposing teeth of the top component B would remain in the key ways D1, D2 of the auger D, thereby locking the position of the auger D relative to the top component B.

The sheath C may also expand as the top component B and the auger D are pulled away from the base component A. Due to such expansion, mucus or other occlusive material within the ETT 400 that is extracted by the auger D may be captured in the sheath C. Once the auger D has been fully extracted from the ETT 400 and is contained in the sheath C (as depicted in FIG. 5), the base component A may be detached from the fitting of the ETT 400, and the lumen clearing device 300 may be discarded.

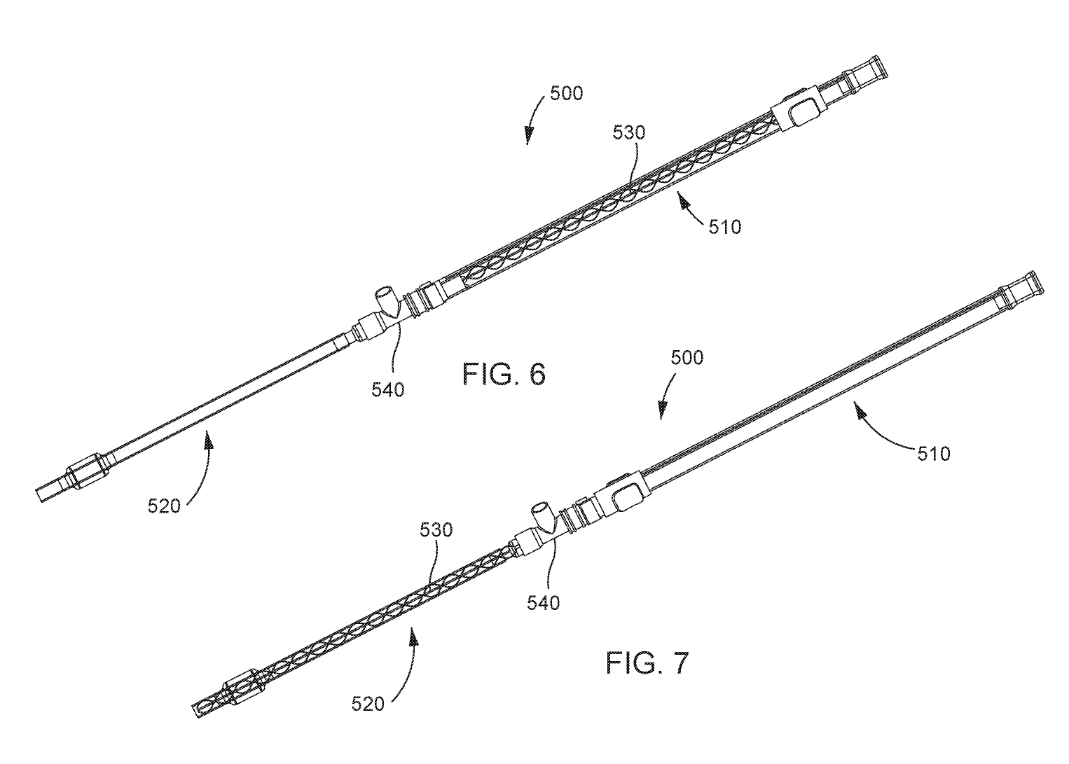

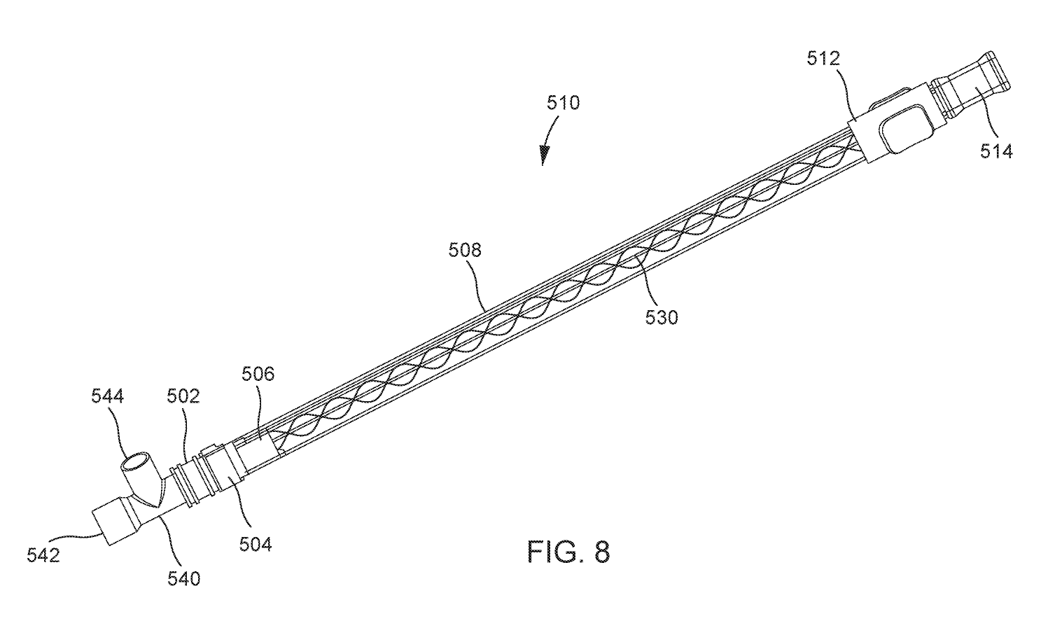

Referring now to FIGS. 6 through 16, an example lumen clearing device 500 is depicted. The lumen clearing device 500 includes a manual advancement device 510. The manual advancement device 510 may be attached to an airway device such as an endotracheal tube 520 using an adaptor portion or tube adaptor 540. The tube adaptor 540 includes a distal end and a proximal end (as shown in FIG. 8). The distal end of the tube adaptor 540 includes an opening 542. The opening 542 may be fitted around a proximal end of the endotracheal tube 520. The tube adaptor 540 also includes a side port 544. The side port 544 may be configured to attach to a source of air such as a ventilation tube. The side port 544 may be disposed at an angle relative to a longitudinal axis of the tube adaptor 540. As depicted in FIG. 8, the side port 544 may be disposed at an angle of 90 degrees relative to the longitudinal axis of the tube adaptor 540. The proximal end of the tube adaptor 540 may be attached a distal end of the manual advancement device 510. Specifically, as described in further detail below, the manual advancement device 510 may include an adaptor portion 502, which may be configured to attach to the distal end of the tube adaptor 540. The proximal end of the tube adaptor 540 may also include an opening. The adaptor portion 502 of the manual advancement device 510 may be designed to connect to the opening at the proximal end of the tube adaptor 520, thereby forming a channel for allowing an auger assembly 530 to travel into the endotracheal tube 520. When the manual advancement device 510 is not attached to the proximal end of the tube adaptor 540, a cap (not shown) may be used to close off the opening at the proximal end of the tube adaptor 540. The cap may prevent containments and other material from entering into the endotracheal tube 520 when the manual advancement device 510 is not attached to the tube adaptor 520 and being used to clean the endotracheal tube 520.

The manual advancement device 510 may be configured to axially displace or advance the auger assembly 530 such that it is advanced into an interior lumen of the endotracheal tube 520. More specifically, the manual advancement device 510 may advance the auger assembly 530 from an initial state (as depicted in FIG. 6) to a deployed state (as depicted in FIG. 7). In its initial state, the auger assembly 530 may be disposed within the manual advancement device 510. In its deployed state, the auger assembly 530 may be disposed in the endotracheal tube 520 such that its distal end is disposed at a distal end of the endotracheal tube 520.

The auger assembly 530 may include a ridge or flute 531 and a shaft 532. The flute 531 may extend in a helical configuration from the shaft 532. The flute 531 may have properties similar to the flute 106, and the shaft 532 may have properties similar to the shaft 104. In particular, the shaft 532 may be formed of a flexible material that is capable of deforming to a curve of the endotracheal tube 520 but is rigid in a torsional direction. As such, the shaft 532 may be formed such that it is flexible along a neutral bending axis but is torsionally rigid. The flute 531 may be of one-piece construction. The flute 531 may have an inner portion that is configured to deform to an internal dimension of the endotracheal tube 520 and an outer portion that is configured to shear against an inner wall of the endotracheal tube 520. The outer portion of the flute 531 may be designed to be sufficiently rigid such that it is capable of making contact with and shearing against an inner wall of the endotracheal tube 520. Thus, the outer portion of the flute 531 may be designed to be more rigid than the inner portion of the flute 531. The flute 531 of the auger assembly 530 may be shaped such that, when it is rotated, it is capable of conveying occlusive material that it contacts in the endotracheal tube 520 in a proximal direction.

FIG. 8 provides a detailed view of the manual advancement device 510 with the auger assembly 530 depicted in its initial state. The manual advancement device 510 may include an adaptor portion or adaptor 502. As described above, the adaptor 502 may be configured to attach to a proximal end of the tube adaptor 540 to provide a passageway or channel for the auger assembly 530 to travel into the endotracheal tube 520. The manual advancement device 510 may also include a separate adaptor portion or adaptor cap 506. The adaptor 502 and the adaptor cap 506 may be locked together by a locking mechanism or adaptor cap lock 504. The adaptor cap lock 504 may be attached to the adaptor cap 506. In an aspect, the adaptor cap lock 504 may be snapped onto the adaptor cap 506.

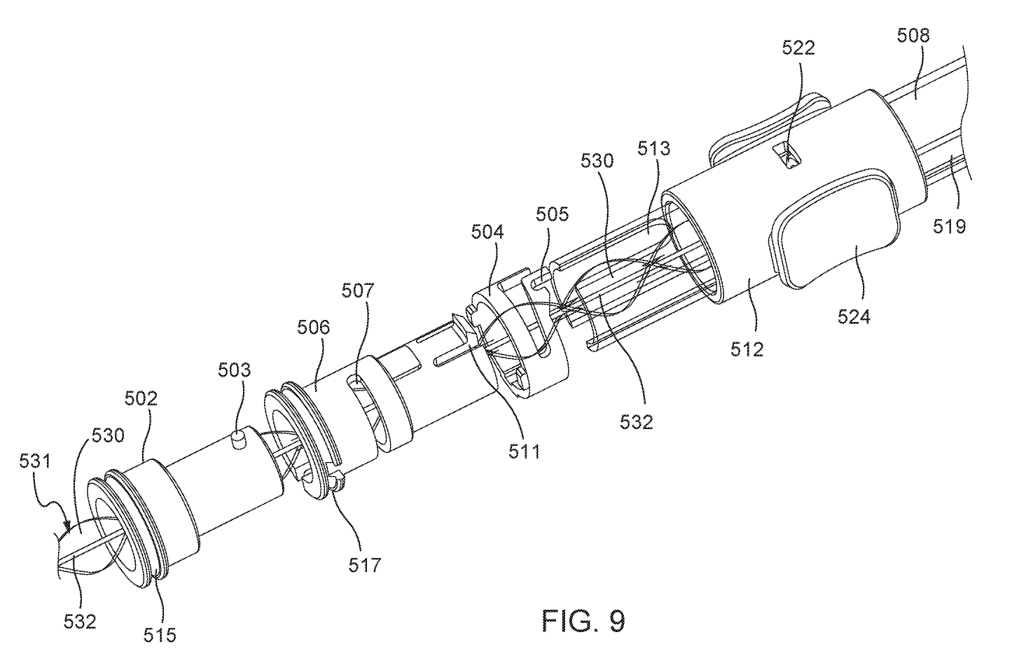

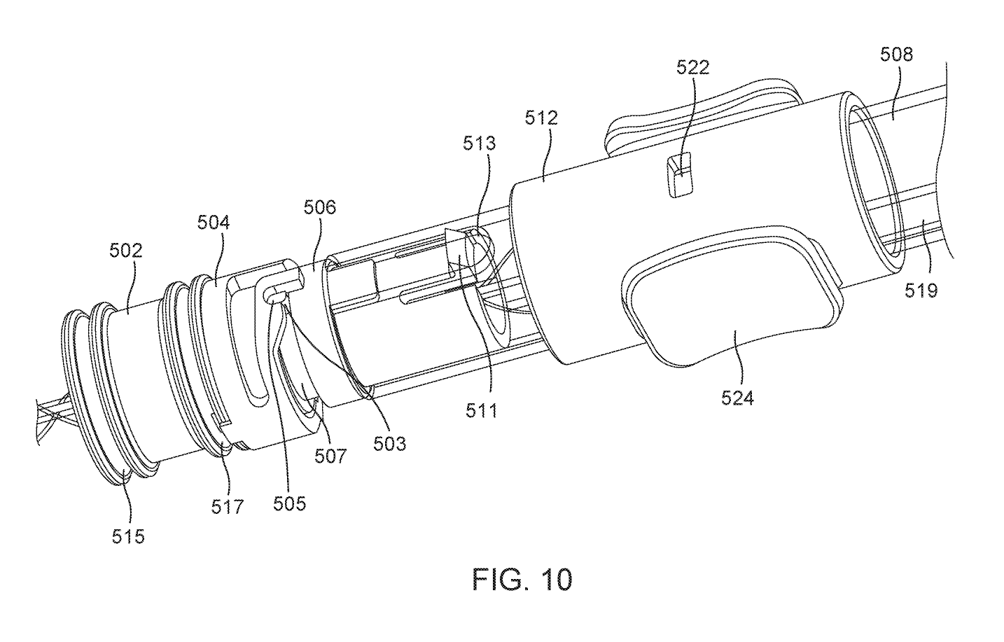

FIG. 9 depicts a deconstructed view of a distal end of the manual advancement device 510, and FIG. 10 depicts an enlarged view of the distal end of the manual advancement device 510. As depicted in FIG. 10, the adaptor 502 may comprise a protrusion such as a key or a boss 503. The boss 503 of the adaptor 502 may be positioned within a slot or channel 507 of the adaptor cap 506 when the adaptor 502 and the adaptor cap 506 are locked together. The adaptor cap lock 504 may include a retaining arm 505 that receives and holds onto the boss 503 of the adaptor 502 such that the adaptor 502 cannot rotate relative to the adaptor cap 506.

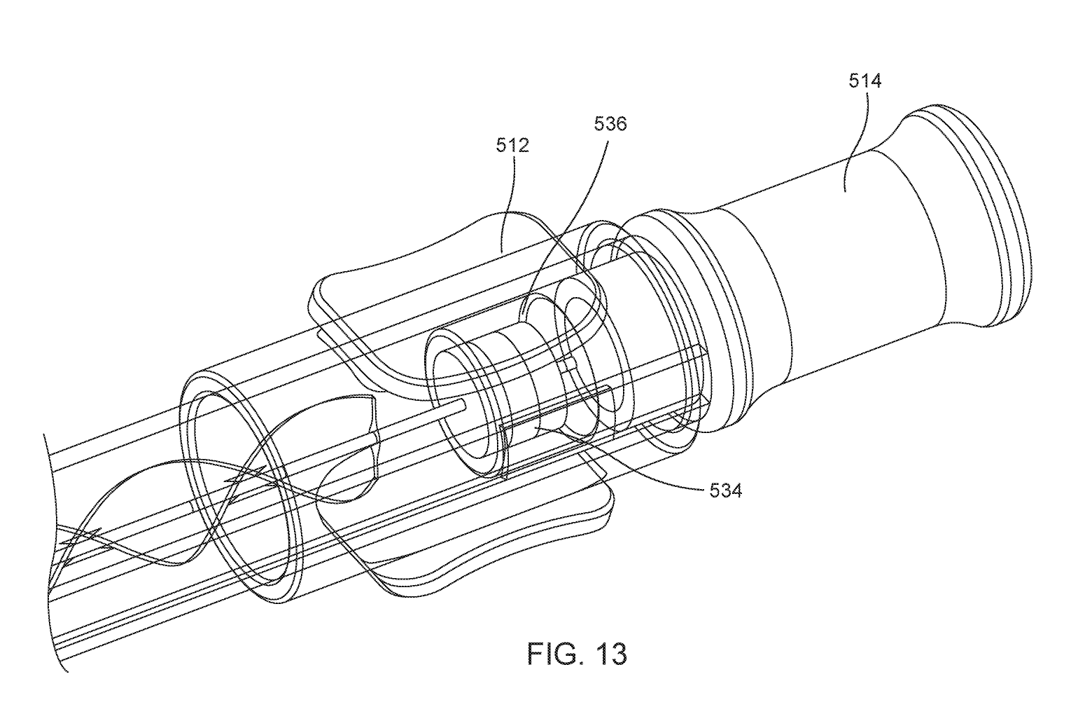

The manual advancement device 510 further includes a tubular portion or advancer tube 508 and an advancer portion or advancer 512. The advancer 512 may be disposed on the advancer tube 508. The advancer 512 may have an outer portion 524 that is disposed on the outside of the advancer tube 508 and an inner portion 536 that is disposed on the inside of the advancer tube 508 (see FIG. 13). The advancer 512 may be configured to axially displace along the advancer tube 508. The advancer tube 508 may have a groove 519 that receives a portion of the advancer 512. The slot 519 may guide the advancer 512 as it displaces along the length of the advancer tube 508 from a proximal end of the advancer tube 508 to a distal end of the advancer tube 508. The slot 519 may also prevent the advancer 512 from rotating as it axially displaces along the advancer tube 508.

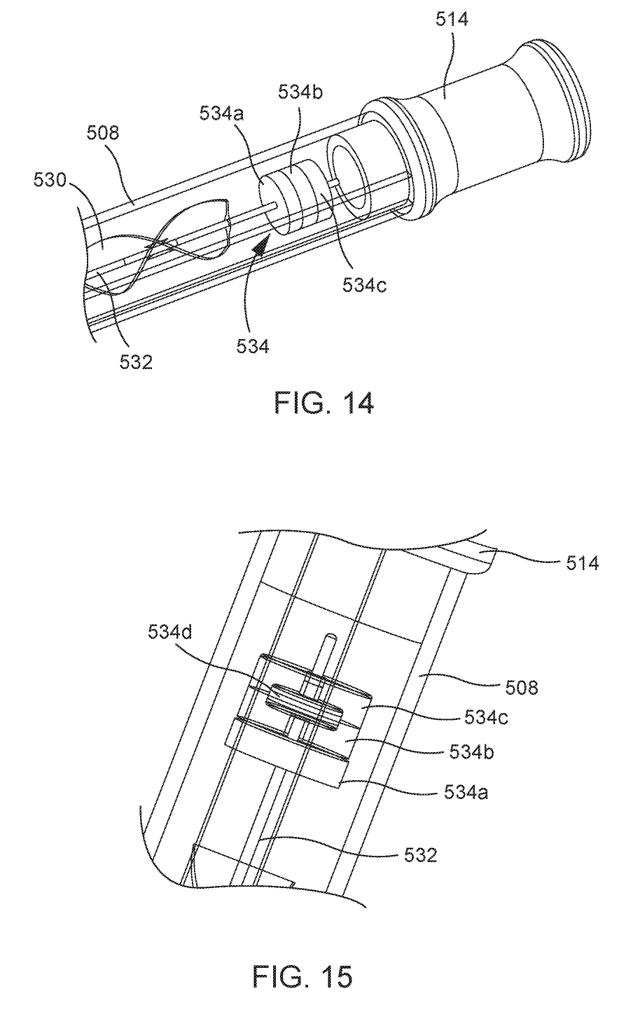

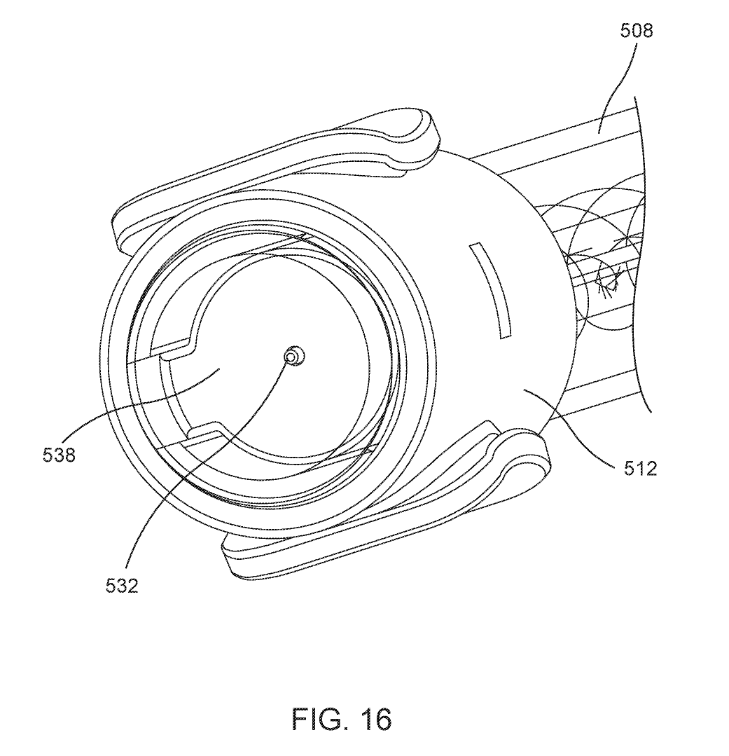

The advancer 512 may be used to insert the auger assembly 530 into the endotracheal tube 520. As depicted in FIG. 13, the advancer 512 (shown in transparent form) may include an inner portion 536 that is configured to receive one or more thrust bearing components 534. The one or more thrust bearing components 534 may include a thrust bearing retainer 534a and two thrust bearings 534b, 534c. See FIGS. 14 and 15. The two thrust bearings 534b, 534c may surround a thrust bearing disk 534d. The thrust bearing disk 534d may be attached to a distal end of the shaft 532 of the auger assembly 530. The two thrust bearings 534b, 534c may be designed to allow the thrust bearing disk 534d to freely rotate about its central axis. In particular, the two thrust bearings 534b, 534c may be made from a material with a low friction coefficient or be coated with a material with a low friction coefficient such that they permit the thrust bearing disk 534d to rotate about its central axis. The thrust bearings 534b, 534c allow the auger assembly 530 to rotate while the advancer 512 advances the auger assembly 530 into the endotracheal tube 520.

As depicted in FIG. 16, the inner portion 536 of the advancer includes a cap or end piece 538. The cap 538 and the thrust bearing retainer 534a surround both sides of the thrust bearings 534b, 534c and the thrust bearing disk 534d, thereby retaining them within the inner portion 536 of the advancer 512. Accordingly, as the advancer 512 is axially displaced along the length of the advancer tube 508, the cap 538 and the thrust bearing retainer 534a move the thrust bearings 534b, 534c and the thrust bearing disk 534d, which in turn advances the auger assembly 530 along the length of the advancer tube 508.

The manual advancement device 510 may also include a handle 514. The handle 514 may be used by an operator to hold onto the manual advancement device 510 and stabilize it in place. The handle 514 may be attached to a proximal end of the advancer tube 508.

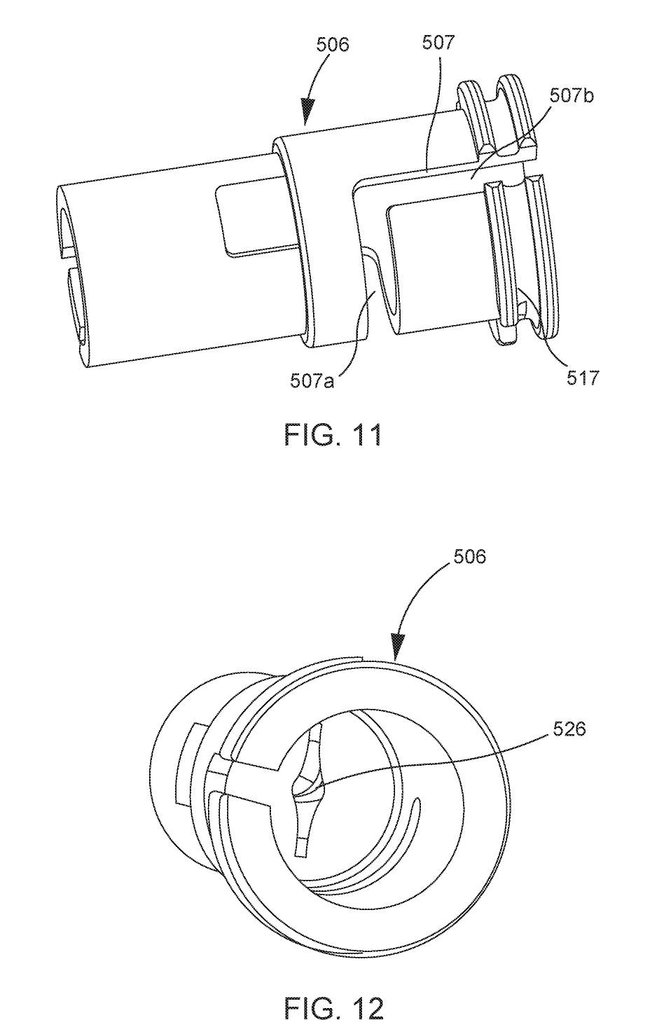

Referring now to FIGS. 11 and 12, enlarged views of the adaptor cap 506 are depicted. As depicted in FIG. 12, the adaptor cap 506 includes a helical groove or opening 526 that is sized to receive the auger assembly. The helical groove 526 may be configured to rotate the auger assembly 530 as it is advanced beyond the adaptor cap 506 and into the endotracheal tube 520. The helical groove 526 may rotate the auger assembly 530 at a rate that is equal to or greater than a rate of advancement of the auger assembly 530 into the lumen of the endotracheal tube 520.

As depicted in FIG. 11, the adaptor cap 506 also includes the channel 507, which guides the separation of the adaptor cap 506 from the adaptor 502. As described above, the adaptor 502 includes a boss 503 that may be received in the channel 507 of the adaptor cap 506 when the adaptor 502 and the adaptor cap 506 are locked together. The channel 507 of the adaptor cap 506 may comprise a curved segment 507a that runs partially around a circumference of the adaptor cap 506 and a straight segment 507b that runs along a longitudinal length of the adaptor cap 506. The curved segment 507a may connect at one of its ends to the straight segment 507b, thereby forming a continuous channel 507. When the adaptor cap 506 is locked onto the adaptor 502, the boss 503 of the adaptor 502 may be disposed in the curved segment 507a of the channel 507 at an end opposite from the end that connects to the straight segment 507b. The adaptor cap lock 504 may lock the boss 503 in this position, thereby preventing the adaptor 502 and the adaptor cap 506 from moving relative to one another.

As depicted in FIG. 10, the adaptor cap 506 also includes a tab 511 that is configured to lock the adaptor cap 506 to the advancer 512 when the advancer 512 is advanced to a final position on the advancer tube 508. The tab 511 of the adaptor cap 506 may snap into an opening 522 in the advancer 512 when a distal portion of the advancer 512 engages with the adaptor cap lock 504. The tab 511 may be disposed in a slot or opening 513 of the advancer tube 508. The engagement of the distal portion of the advancer 512 with the adaptor cap lock 504 may displace the retaining arm 505 of the adaptor cap lock 504, thereby releasing the boss 503 of the adaptor 502. When the boss 503 is released, the adaptor cap 506 may be rotated or turned relative to the adaptor 502. The rotation of the adaptor cap 506 relative to the adaptor 502 may move the boss 503 along the curved segment 507a of the adaptor cap 506. Once the boss 503 is moved to the end of the curved segment 507a that connects with the straight segment 507b, the adaptor cap 506 may be pulled and removed from the adaptor 502. In other words, after the boss 503 is released from the adaptor cap lock 504, the adaptor cap 506 may be turned a number of degrees (e.g., 180 degrees) relative to the adaptor 502 and then separated from the adaptor 502. The rotation or turning of the adaptor cap 506 may rotate the auger assembly 530, thereby allowing a distal end of the auger assembly 530 to sweep around its final position in the endotracheal tube 520. The distal end of the auger assembly 530 may be disposed at a distal end of the endotracheal tube 520. Therefore, the rotation of the adaptor cap 506 may rotate the auger assembly 530, allowing it to sweep and capture any remaining material (e.g., mucus, sputum) that is disposed at the distal end of the endotracheal tube 520.

To remove the auger assembly 530 from the endotracheal tube 520, the adaptor cap 506 may be separated from the adaptor 502 and pulled in a proximal direction away from the endotracheal tube 520. The manual advancement device 510 may include a sheath (not depicted) that is held between the adaptor 502 and the adaptor cap 506. As depicted in FIGS. 9 and 10, the adaptor 502 may include a groove 515 for receiving a fastener that is configured to fasten a first end of the sheath to the adaptor 502. And the adaptor cap 506 may include a groove 517 for receiving a fastener that is configured to fasten a second end of the sheath to the adaptor 506. Example fasteners include O-rings or other types of mechanical fasteners. The fasteners may clamp down on the ends of the sheath, thereby holding them in place on the adaptor 502 and the adaptor cap 506. The sheath may initially be held in a bunched or collapsed state between the adaptor 502 and the adaptor cap 506. But when the adaptor cap 506 is separated from the adaptor 502, the sheath may expand to capture the auger assembly 530 and any material that the auger assembly removes from the endotracheal tube 520. When the auger assembly 530 is removed from the endotracheal tube 520, the adaptor 502 may be detached from the tube adaptor 540. The manual advancement device 510 and the auger assembly 530 may be disposed of, and the opening at the proximal end of the tube adaptor 540 may be covered again using an end cap.

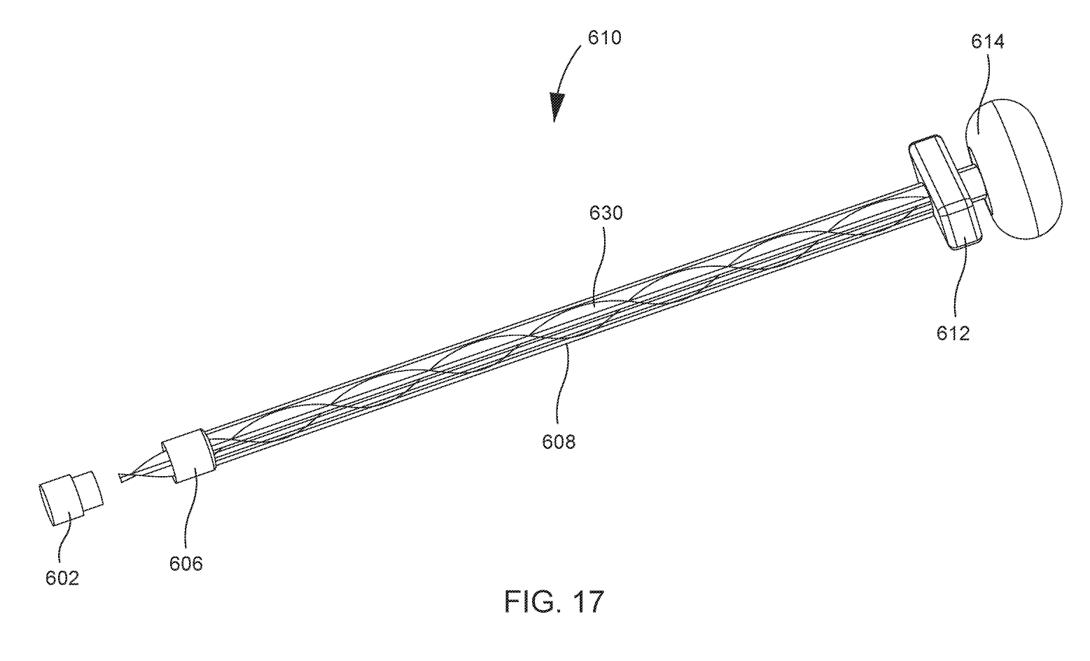

Referring now to FIGS. 17 through 19, another example of a manual advancement device is depicted. The manual advancement device 610 includes a first adaptor portion or adaptor 602, a second adaptor portion or adaptor cap 606, a tubular portion or advancer tube 608, an advancer portion or advancer 612, and a handle 614. The adaptor 602, the adaptor cap 606, the advancer tube 608, the advancer 612, and the handle 614 function similarly to the corresponding components in the example manual advancement device 510. The manual advancement device 610 may be designed to axially displace or advance an auger assembly 630 into an interior lumen of an endotracheal tube such as the endotracheal tube 520. The auger assembly 630 may have the same construction as the auger assembly 530.

The handle 614 of the manual advancement device 610 may be used by an operator to hold onto the manual advancement device 610 and stabilize it in place. The handle 614 may have a round knob-like configuration.

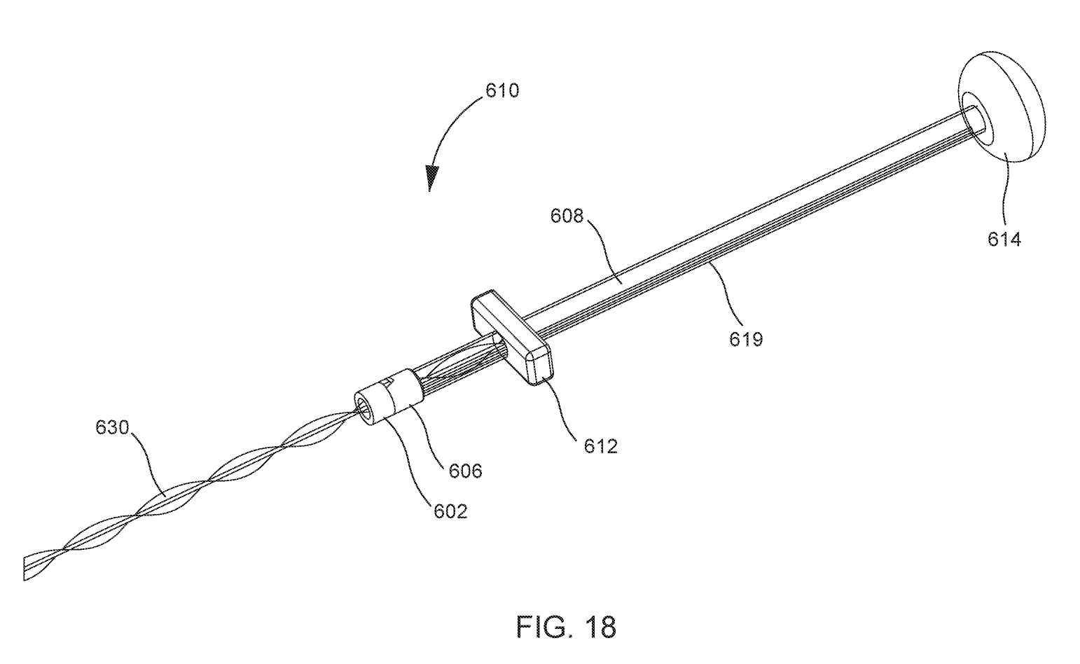

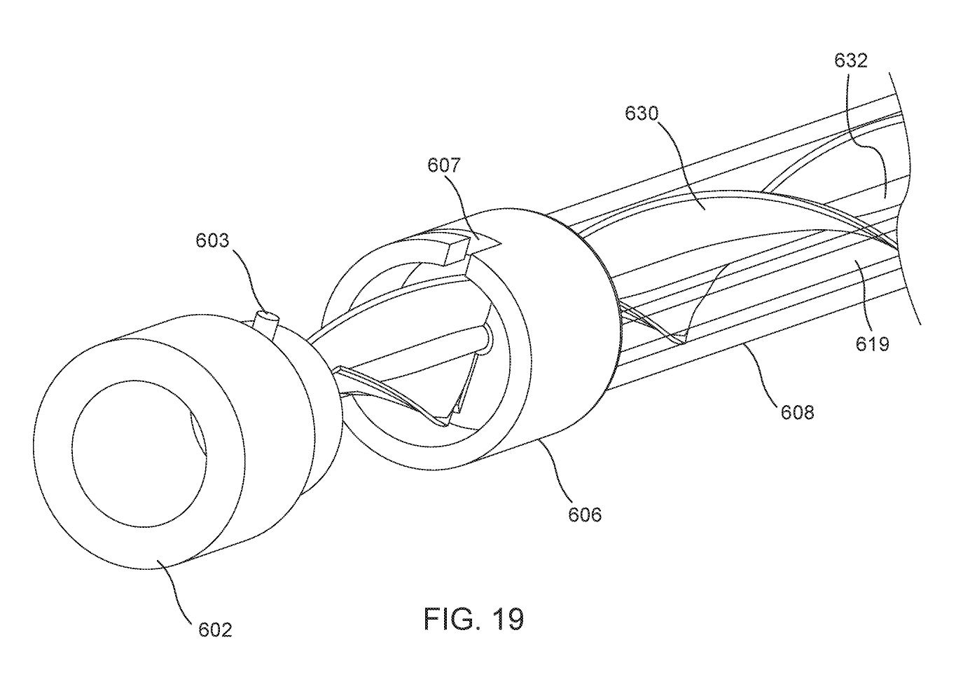

The adaptor 602 may be configured to attach to a tube adaptor such as the tube adaptor 540 described above. As depicted in FIG. 18, the adaptor 602 may engage with the adaptor cap 606, similar to the engagement of the adaptor 502 and the adaptor cap 506. FIG. 19, which provides an enlarged view of the adaptor 602 and the adaptor cap 606 shows that the adaptor 602 may include a protrusion or boss 603 that fits into a channel or slot 607 on the adaptor cap 606. The channel 607 may include a straight segment and a curved segment, which are joined together to form a continuous channel. The boss 603 of the adaptor 602 may initially be positioned at an end of the curved segment of the channel 607 opposite from the end that connects to the straight segment of the channel 607.

The adaptor cap 606 may include a helical opening that is capable of rotating the auger assembly 630 as it is advanced through the adaptor cap 606 and into the interior lumen of the endotracheal tube. See FIG. 19. The advancer 612 may be used to advance the auger assembly 630 into the lumen of the endotracheal tube. The advancer 612 may be disposed on the advancer tube 608. The advancer 612 may have a rectangular shape, such as that depicted in FIGS. 17 and 18. The manual advancement device 610, similar to the manual advancement device 510, may include thrust bearings, a thrust bearing retainer, and a thrust bearing disk (not depicted). The thrust bearings, a thrust bearing retainer, and a thrust bearing disk may effectively attach the auger assembly 630 to the advancer 612 but allow the auger assembly 630 to rotate as it is advanced through the adaptor cap 606 and into the endotracheal tube.

The advancer 612 may be advanced a predetermined distance along the advancer tube 608. See FIG. 18. The advancer tube 608 may include markings or some other type of indication for showing the distance that the auger assembly 630 has been advanced into the endotracheal tube. When the auger assembly 630 has been advanced such that its distal end is disposed at the distal end of the endotracheal tube, the adaptor cap 606 may be separated from the adaptor 602. To separate the adaptor cap 606 from the adaptor 602, the adaptor cap 606 may be rotated a certain number of degrees relative to the adaptor 602 and removed from the adaptor 602. Similar to the manual advancement device 510, the manual advancement device 610 may have a sheath (not depicted) that is held in place between the adaptor 602 and the adaptor cap 604. When the adaptor cap 604 is separate from the adaptor 602, this sheath may expand to capture the auger assembly 630 as it is being extracted from the endotracheal tube. The sheath may also capture any material that the auger assembly 630 removes from the lumen of the endotracheal tube. When the auger assembly 630 has been removed, the adaptor 602 may be detached from the tube adaptor, and the manual advancement device 610 and the auger assembly 630 may be discarded.

The many features and advantages of the lumen clearing devices and manual advancement devices described herein are apparent from the detailed specification, and thus, the claims cover all such features and advantages within the scope of this application. Further, numerous modifications and variations are possible. As such, it is not desired to limit the lumen clearing device to the exact construction and operation described and illustrated and, accordingly, all suitable modifications and equivalents may fall within the scope of the claims.

1.-22. (canceled)

23. A method of clearing a lumen of an airway device, comprising:
inserting a distal portion of a lumen clearing device into the lumen of the airway device, the distal portion of the lumen clearing device comprising a shaft and a ridge extending from the shaft in a spiral configuration; and
rotating the shaft and the ridge of the lumen clearing device to capture and convey a material disposed within the lumen of the airway device out of the lumen of the airway device,
wherein the airway device is at least one of an endotracheal tube, a tracheostomy tube, and a laryngeal mask airway tube.

24. The method of claim 23, wherein the shaft and the ridge of the lumen clearing device are rotated at a rate equal to or greater than a rate of spiral advancement of the ridge along an interior wall of the lumen.

25. The method of claim 23, further comprising capturing the material conveyed out of the lumen of the airway device using a sheath attached to the lumen clearing device.

26. (canceled)

27. A method of clearing a lumen of an airway device, comprising:
attaching a manual advancement device to a proximal end of the airway device, the manual advancement device comprising:
a tubular portion housing an auger assembly, the auger assembly comprising a shaft and a ridge extending from the shaft in a helical configuration;
an advancer portion disposed on the tubular portion and configured to slide along the tubular portion and to insert the auger assembly into the lumen of the airway device; and
a first adaptor portion configured to rotate an auger assembly;
sliding the advancer portion along the tubular portion to insert the auger assembly into the lumen of the airway device,
wherein the auger assembly is configured to rotate when the advancer portion when the advancer portion inserts the auger assembly into the lumen of the airway device,
wherein the auger assembly when rotated is configured to contact and convey a material disposed in the lumen of the airway tube out of the lumen of the airway tube, and
wherein the airway device is at least one of an endotracheal tube, a tracheostomy tube, and a laryngeal mask airway tube.

28. The method of claim 27, wherein attaching the manual advancement device to the proximal end of the airway device comprises attaching a distal end of a second adaptor portion to the proximal end of the airway device.

29. The method of claim 28, further comprising supplying a source of air to the airway device through a port disposed on the second adaptor portion.

30. The method of claim 27, further comprising capturing the material conveyed out of the lumen of the airway device using a sheath attached to the manual advancement device.

31. The method of claim 30, wherein the sheath comprises a first portion attached to the first adaptor portion and a second portion attached to a third adaptor portion.
